(19) United States
(12) Patent Application Publication
(10) Pub. No.: US 2005/0131413 A1
(43) Pub. Date: Jun. 16, 2005

(54) Bone plate with interference fit screw

(76) Inventors: Shawn W. O'Driscoll, Rochester, MN (US); Joel Gillard, Portland, OR (US)

Correspondence Address: KOLISCH HARTWELL, P.C., 520 S.W. YAMHILL STREET, SUITE 200, PORTLAND, OR, 97204, US

(21) Appl. No.: 10/873,410

(22) Filed: Jun. 21, 2004

Related U.S. Application Data
(60) Provisional application No. 60/480,517, filed on Jun. 20, 2003.
(60) Provisional application No. 60/498,807, filed on Aug. 28, 2003.
(60) Provisional application No. 60/498,870, filed on Aug. 28, 2003.

(51) Int. Cl. A61B 17/56

(52) U.S. Cl. 606/73

(57) ABSTRACT

Systems, including apparatus and methods, for internal fixation of a fractured or otherwise compromised bone. These systems may include and/or make use of bone plates, locking screws, and/or kits, among others.

### CROSS-REFERENCES TO PRIORITY APPLICATIONS

This application is based upon and claims the benefit under 35 U.S.C. § 119(e) of the following U.S. provisional patent application, which is incorporated herein by reference in its entirety for all purposes: Ser. No. 60/480,517, filed Jun. 20, 2003.

### CROSS-REFERENCES TO RELATED APPLICATIONS

This application incorporates by reference in their entirety for all purposes the following patent applications: U.S. Provisional Patent Application Ser. No. 60/297,008, filed Jun. 8, 2001; PCT patent application Ser. No. PCT/US02/18623, filed Jun. 10, 2002; and U.S. patent application Ser. No. 10/731,173, filed Dec. 8, 2003.

This application also incorporates by reference in their entirety for all purposes the following patent applications: U.S. Provisional Patent Application Ser. No. 60/498,807, filed Aug. 28, 2003; and U.S. Provisional Patent Application Ser. No. 60/498,870, filed Aug. 28, 2003.

### INTRODUCTION

The human skeleton is composed of 206 individual bones that perform a variety of important functions, including support, movement, protection, storage of minerals, and formation of blood cells. These bones can be grouped into two categories: the axial skeleton, and the appendicular skeleton. The axial skeleton consists of 80 bones that make up the body's center of gravity, and the appendicular skeleton consists of 126 bones that make-up the body's appendages. The axial skeleton includes the skull, vertebral column, ribs, and sternum, among others, and the appendicular skeleton includes the long bones of the upper and lower limbs, and the clavicles and other bones that attach these long bones to the axial skeleton, among others.

To ensure that the skeleton retains its ability to perform its important functions, and to reduce pain and disfigurement, fractured bones should be repaired promptly and properly. Typically, fractured bones are treated using fixation devices, which reinforce the fractured bone and keep it aligned during healing. Fixation devices may take a variety of forms, including casts for external fixation and bone plates for internal fixation, among others. Casts are minimally invasive, allowing reduction and fixation of simple fractures from outside the body. In contrast, bone plates are sturdy internal devices, usually made of metal, that mount directly to the bone adjacent the fracture.

Bone plates may be used to repair a fracture, as follows. First, a surgeon selects an appropriate plate. Second, the surgeon reduces (sets) the fracture. Finally, the surgeon fastens the plate to opposite sides of the fracture using suitable fasteners, such as screws and/or wires, so that the bone is fixed in position. The mounted plate may be left in place permanently, or it may be removed after the bone has healed sufficiently.

Bone plates typically include a plurality of apertures for receiving fasteners such as bone screws. These apertures may be unthreaded or threaded. Unthreaded apertures may be used to provide compression. Specifically, a bone screw inserted through an unthreaded aperture will thread only into the bone. Thus, the screw Will turn without limitation until the plate and bone are brought into contact, or compressed. Furthermore, variable-angle screws may be used in conjunction with unthreaded apertures. This may allow the angle with which the screw enters the bone to be chosen by the surgeon intraoperatively.

Threaded apertures, in contrast, may be used to leave a space or gap between the plate and bone. Specifically, a bone screw inserted through a threaded aperture will thread into both the plate and bone. Thus, there will be a space between the plate and bone if the screw is threaded fully into the plate (such that the screw cannot turn any more) before compression occurs between the bone and the plate. The space may have several advantages, such as facilitating healing by preserving blood flow to the bone, and, in the case of a removable bone plate, reducing undesirable bonding of the plate to the bone. However, a threaded aperture typically limits the angle of installation of the screw, since the threads provide a fixed orientation for engagement between the screw and the aperture. For this reason, screws configured to engage with threaded apertures are sometimes called “fixed-angle” screws.

Bone plates are provided with unthreaded or threaded apertures based on the best guesses of manufacturers regarding the most suitable apertures for a given indication. Thus, surgeons currently may be limited to using plates designed only for the most common fractures. However, although many fractures share common motifs, no two fractures or bones are identical. In some cases, a surgeon may want a threaded aperture where an unthreaded aperture is provided, or vice versa. Similarly, a surgeon may find it desirable to insert a variable-angle screw through a threaded aperture, or to insert a fixed-angle screw through an unthreaded aperture.

### SUMMARY

The present teachings provide systems, including apparatus and methods, for internal fixation of a fractured or otherwise compromised bone. These systems may include and/or make use of bone plates, locking screws, and/or kits, among others.

### BRIEF DESCRIPTION OF THE DRAWINGS

FIG. 1 is a side view of an exemplary locking screw that is configured to lock into place at a fixed angle within an unthreaded aperture of a bone plate, in accordance with aspects of the present teachings.

FIG. 2 is a partially cross-sectional side view of an exemplary bone plate, including an unthreaded aperture configured to receive a locking screw at a fixed angle, in accordance with aspects of the present teachings.

FIG. 3 is a partially cross-sectional side view of the locking screw of FIG. 1 engaged with the unthreaded aperture in the bone plate of FIG. 2.

FIG. 4 is a partially cross-sectional side view of a non-locking screw engaged with the unthreaded aperture in the bone plate of FIG. 2.

### DETAILED DESCRIPTION

The present teachings provide systems, including apparatus and methods, for internal fixation of a fractured or otherwise compromised bone. These systems may include and/or make use of locking screws and/or bone plates, among others. The locking screws may lock into place at a fixed angle within an unthreaded aperture of the plate, for example, due to interference between one or more threads of the screw and an inner lip of the aperture. Thus, a locking screw may provide spaced engagement of an aperture to the underlying bone. Apertures of the plate also may be configured to accept non-locking, variable-angle screws that may be inserted through the apertures at variable angles to provide compressed engagement of the aperture to the bone. Thus, the systems may allow a surgeon to choose independently between compressed and spaced engagement for each aperture in the plate, by independently selecting a locking or a non-locking screw for each unthreaded aperture of the plate.

### I. EXEMPLARY LOCKING SCREW/BONE PLATE SYSTEM

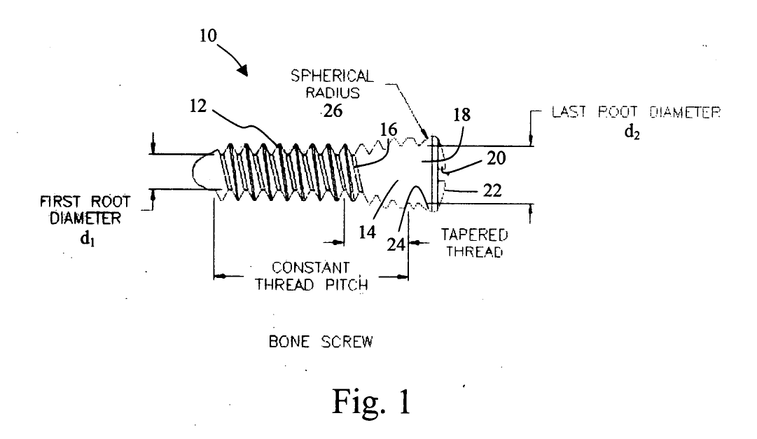

FIG. 1 shows a diagram of a locking screw 10, in accordance with aspects of the present teachings. Screw 10 includes a shaft portion 12, and a head portion 14. The shaft portion may be unthreaded, continuously threaded as depicted in FIG. 1, or discontinuously threaded. The shaft portion may have a maximum root diameter (i.e., a shaft diameter not including the threads) d₁. The head portion also may be threaded, and tapered so that the root diameter increases from d₁ at a proximal end 16 of the head portion, to d₂ (d₂>d₁) at a distal end 18 of the head portion. The locking screw may include a slot 20, at a distal surface 22 of the head portion, to receive a screwdriver blade. More generally, surface 22 may be cannulated or otherwise configured to receive an Allen wrench, a screwdriver blade, and/or any other device or driver (including a specialized tool) suitable for exerting torque on the screw to turn it.

When the shaft portion of screw 10 is threaded, the distance between threads (thread pitch) may be variable or, as depicted in FIG. 1, constant along most of the length of screw 20. However, a circular groove 24 is provided at distal end 18 of the head portion. The groove extends around the circumference of the screw, and is equidistant from distal surface 22. That is, the groove 24 is circular, rather than helical as are the other threads of the screw. The head portion of the screw also may include an arcuate portion or spherical radius 26, disposed between groove 24 and distal surface 22. Groove 24 and spherical radius 26 are configured to engage securely with an aperture of a bone plate, as described below.

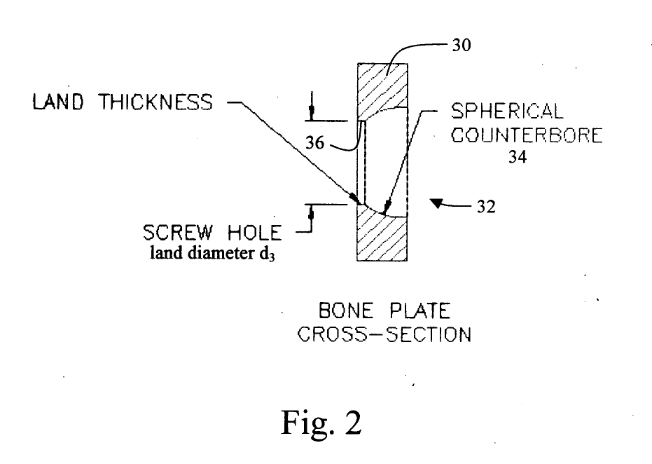

FIG. 2 shows a partially cross sectional view of a bone plate 30, including an aperture 32 configured to receive a locking screw, such as screw 10 of FIG. 1. Aperture 32 includes an arcuate counterbore portion 34, and a land (or lip) portion 36. Counterbore portion 34 may be shaped to match the arcuate surface of head portion 14 of the screw, so that the screw will seat securely within the aperture. Thus, in FIG. 2, the counterbore is spherical, to match spherical radius 26 of the head portion of the screw. In the depicted embodiment, land portion 36 has a diameter d₃ that is approximately equal to, or slightly greater than, the largest root diameter d₂ of screw 10. Thus, the land portion may be configured to fit within groove 24, to secure the screw within the aperture.

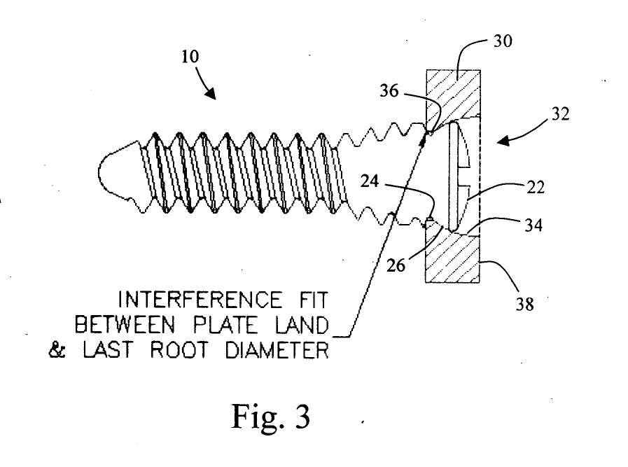

FIG. 3 shows screw 10 seated within aperture 32. Spherical radius 26 of the screw may fit securely within counterbore portion 34 of the aperture. At the same time, land portion 36 of the aperture may fit within groove 24 of the screw, possibly compressing the land portion within the groove. This compression may lead to slight deformations of the land portion and/or the groove, and may result in a relatively large amount of friction between the land portion and the groove. This can have the effect of locking the screw into place at a fixed angle within the aperture. In other words, an interference fit may occur between the aperture and the screw, and particularly between land portion 36 and groove 24.

When screw 10 is fully inserted and seated within aperture 32, distal surface 22 of the head of the screw is disposed below an outer surface 38 of bone plate 30, as can be seen in FIG. 3. That is, the head of the screw is recessed within the aperture, and does not protrude above surface 38 of the bone plate. This may ameliorate pain and discomfort sometimes suffered by a patient, if a screw head and/or a head portion of another fastener protrudes above the surface of the plate. A non-locking, variable-angle fastener also may be inserted through aperture 32, with the head of the fastener similarly recessed within the aperture, as described below.

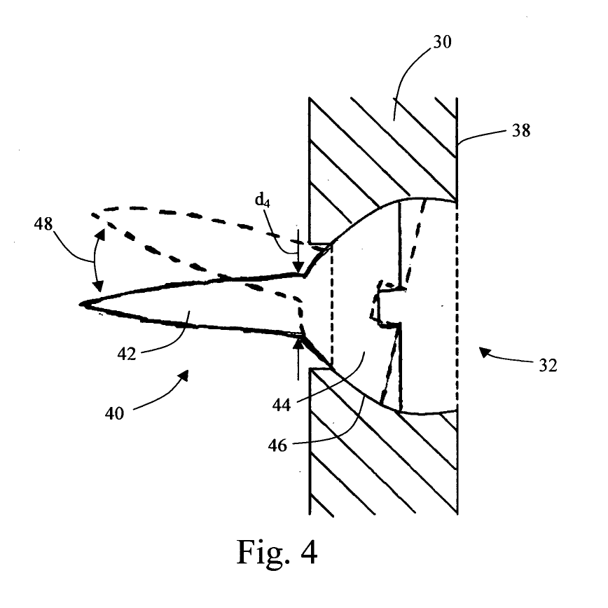

FIG. 4 shows a variable-angle bone screw 40 disposed within aperture 32 of bone plate 30. Screw 40 has a shaft portion 42, and an unthreaded head portion 44. Shaft portion 42 is depicted as unthreaded, but in general may be threaded, partially threaded, or unthreaded. The head portion of screw 40 may include an arcuate engagement surface 46, which may be pivotable within aperture 32 to facilitate varying the angle with which the screw enters the bone, as indicated by arrow 48. Shaft portion 42 has a maximum diameter d₄ that is nominally less than the diameter d₃ of the aperture, so that the shaft of the screw may pass freely through the aperture without engaging or interfering with land portion 36 of the aperture. Instead, the threads (or in general, the shaft) of screw 40 engage only with the underlying bone, compressing plate 30 against the bone.

As in the case of a locking screw, the head of variable-angle screw 40 may be recessed within aperture 32, so that the screw does not protrude above surface 38 of the bone plate. Thus, either a locking screw or a variable-angle screw may be inserted through the aperture in order to attach bone plate 30 to the underlying bone, without bumps or protrusions extending above surface 38. This has the advantage that a surgeon may independently select either a locking (i.e., fixed-angle) fastener, or a non-locking (i.e., variable-angle) fastener for each aperture of the plate, to selectively provide spaced or compressed engagement of each aperture to the plate.

The use of locking bone screws such as screw 10, in conjunction with one or more apertures of a bone plate, may allow at least a portion of the plate to be optionally positioned away from the bone. This may allow the periosteum, neurovascular bundle, and the like, to pass under the plate without being pinched or damaged, possibly promoting faster healing of the fractured bone. Positioning the plate slightly away from the bone further may allow for some amount of natural settling and/or thickening (e.g., through calcification) of the fractured bone.

Preshaping the plates allows the inner or bone-facing surface of the plate to follow and substantially match the three-dimensional contour of a bone, along the length of the plate and/or across the width of the plate. For example, the plates may include curved, bent, twisted, and/or tubular inner surfaces that are adapted to face bone and to guide the plates to set onto the bones, initially to enhance fixation and/or to template reduction of bone, and subsequently to increase stability, by grabbing and holding bone fragments. In some embodiments, the plates may be somewhat undercontoured along their long axes, for example, to accommodate soft tissue between a portion of the plate and the bone, or to allow additional custom contouring pre- or peri-operatively, among others.

An aperture configured to accept a locking bone screw may be used to position at least a portion of a bone plate away from the bone, as described above, so that the periosteum, neurovascular bundle, and the like, may pass under the plate without being pinched or damaged. In addition, the plates may include spacing members for this purpose, such as prongs or other projections. Spacing members may be configured to project generally orthogonal to a proximal surface of bone, when the plates are attached in their intended orientation to bone. Accordingly, spacing members may project from the sides and/or bone-facing surfaces of bone plates in a substantially orthogonal direction relative to a plane defined locally by length and width of a bone plate. Alternatively, or in addition, spacing members such as narrow and/or sharp prongs may be used to grasp the bone for increased fixation, in conjunction with and/or independent of additional fixation mechanisms.

### II. EXAMPLES

The following examples describe exemplary fixation systems, in accordance with aspects of the present teachings. These examples are included for illustration and are not intended to limit or define the entire scope of the present teachings.

### II.A EXEMPLARY EMBODIMENTS

The systems provided by the present teachings may be provided in any suitable form, including locking screws and bone plates, among others. Alternatively, or in addition, the systems may include kits, or combinations of materials, for internal fixation of fractured bones. These kits may include plates, fasteners such as locking and/or non-locking bone screws, and/or instructions, among others. The plates provided in a kit may include one or more apertures suitable for receiving a locking screw, among others. These plates and/or fasteners, among others, may be sized and/or shaped to conform to particular regions of bone, or to different portions of the same region of bone, among others. The plates, in particular, may be preshaped (preformed) to fit an average target anatomy, such as a population-averaged shape of a particular anatomical region. The average anatomy may be a human (or other animal) anatomy averaged over any suitable set, such as, for example, adults, adult males, adult females, people that fall within a particular size range, children of a given age, and so on.

### II.B EXEMPLARY INDICATIONS

The systems provided by the present teachings may be used for any suitable indications, including, but not limited to, those described in the patent applications listed above under Cross-References and incorporated herein by reference in their entirety for all purposes. These indications may include combining any feature(s) disclosed herein with any feature(s) described in the listed patent applications, including, but not limited to, the use of a locking screw and/or plate aperture, as described herein, with bone plates, as described therein.

### II.C MISCELLANEOUS EXAMPLES

The following examples describe selected aspects and embodiments of the present teachings, as a series of ordered paragraphs.

1. A bone plate for fixing a discontinuity in a bone, comprising:
a body portion having a bone-facing surface; and
at least one unthreaded aperture, defined by the body portion, for receiving a bone screw, the aperture including:
   a lip portion adjacent the bone-facing surface of the bone plate and configured to frictionally engage a circular groove of the bone screw; and
   an arcuate seating surface shaped to engage an arcuate head portion of the bone screw.

2. The bone plate of paragraph 1, further comprising at least two unthreaded apertures, each aperture having a lip portion and an arcuate seating surface as described in paragraph 1.

3. The bone plate of paragraph 1, wherein the aperture is configured to receive a locking bone screw or a non-locking bone screw.

4. The bone plate of paragraph 3, wherein the lip portion further is configured to engage the locking screw at a fixed angle within the aperture.

5. The bone plate of paragraph 3, wherein the lip portion and the seating surface further are configured to engage the non-locking bone screw at a variable angle within the aperture.

6. The bone plate of paragraph 1, wherein the body portion further comprises:
a bridge portion for spanning the discontinuity in the bone; and
an anchor portion for affixing the bone plate to the bone.

7. The bone plate of paragraph 6, wherein the unthreaded aperture is located in the anchor portion.

8. The bone plate of paragraph 6, wherein the anchor portion further includes attachment structure configured to receive a wire that helps to affix the bone plate to the bone.

9. The bone plate of any of paragraphs 1 to 8, wherein the bone plate is composed of a biocompatible material such as titanium or stainless steel.

10. A bone screw, comprising:
a shaft portion for engaging the bone; and
a head portion having a circular groove for engaging an aperture in a bone plate, the narrowest diameter of the circular groove being smaller than the diameter of the bone screw to either side of the circular groove.

11. The bone screw of paragraph 10, wherein the shaft portion and/or head portion includes threads that terminate at the circular groove.

12. The bone screw of paragraph 11, wherein the head portion is tapered to form a frustoconical section.

13. The bone screw of paragraph 11, wherein the head portion includes an arcuate seating surface for further engaging the aperture.

14. The bone screw of paragraph 12, wherein the arcuate seating surface is shaped to form a spherical radius.

15. The bone screw of paragraph 13, wherein the arcuate seating surface is shaped to be complementary to a seating surface of the aperture.

16. The bone screw of paragraph 11, wherein the circular groove lies in a plane substantially perpendicular to a long axis of the screw.

17. The bone screw of paragraph 16, wherein the threads have a constant thread pitch.

18. The bone screw of paragraph 11, wherein the circular groove engages the aperture by compressing a lip portion of the aperture within the groove.

19. The bone screw of any of paragraphs 10 to 18, wherein the bone screw is composed of a biocompatible material such as titanium or stainless steel.

20. A kit for repairing a discontinuity in a bone, comprising:
a bone plate according to any of paragraphs 1 to 9; and
at least one bone screw according to any of paragraphs 10 to 19, wherein the bone screw is capable of locking engagement with the bone plate.

21. The kit of paragraph 20, further comprising at least one non-locking bone screw.

22. The kit of paragraph 20, further comprising a driver configured to turn the bone screws into the bone and/or bone plate.

23. The kit of paragraph 20, further comprising a clamp configured to hold the bone plate to the bone during installation of the bone plate.

24. The kit of paragraph 20, further comprising instructions regarding use of the bone plate and bone screws.

25. A method of fixing a discontinuity in a bone, comprising:
reducing the discontinuity;
selecting a bone plate according to any of paragraphs 1 to 9, or from the kit of any of paragraphs 20 to 24;
selecting a bone screw according to any of paragraphs 10 to 19, or from the kit of any of paragraphs 20 to 24; and
fastening the bone plate to the bone to stabilize the reduced discontinuity using the bone plate and the bone screw.

26. The method of paragraph 25, wherein the steps of reducing the discontinuity, selecting a bone plate, and selecting a bone screw may be performed in any order.

The disclosure set forth above may encompass multiple distinct inventions with independent utility. Although each of these inventions has been disclosed in its preferred form(s), the specific embodiments thereof as disclosed and illustrated herein are not to be considered in a limiting sense, because numerous variations are possible. The subject matter of the inventions includes all novel and nonobvious combinations and subcombinations of the various elements, features, functions, and/or properties disclosed herein. The following claims particularly point out certain combinations and subcombinations regarded as novel and nonobvious. Inventions embodied in other combinations and subcombinations of features, functions, elements, and/or properties may be claimed in applications claiming priority from this or a related application. Such claims, whether directed to a different invention or to the same invention, and whether broader, narrower, equal, or different in scope to the original claims, also are regarded as included within the subject matter of the inventions of the present disclosure.

1. A system for fixing a discontinuity in a bone, comprising:
a bone plate, comprising:
(a) a body portion having a bone-facing surface; and
(b) at least one unthreaded aperture, defined by the body portion, for receiving a bone screw, the aperture including (i) a lip portion adjacent the bone-facing surface of the bone plate and configured to frictionally engage a circular groove of the bone screw, and (ii) an arcuate seating surface shaped to engage an arcuate head portion of the bone screw.

2. The system of claim 1, further comprising at least two unthreaded apertures, each aperture having a lip portion and an arcuate seating surface as described in claim 1.

3. The system of claim 1, wherein the aperture is configured to receive a locking bone screw or a non-locking bone screw.

4. The system of claim 3, wherein the lip portion further is configured to engage the locking screw at a fixed angle within the aperture.

5. The system of claim 3, wherein the lip portion and the seating surface further are configured to engage the non-locking bone screw at a variable angle within the aperture.

6. The system of claim 1, wherein the body portion further comprises:
a bridge portion for spanning the discontinuity in the bone; and
an anchor portion for affixing the bone plate to the bone.

7. The system of claim 6, wherein the unthreaded aperture is located in the anchor portion.

8. The system of claim 6, wherein the anchor portion further includes attachment structure configured to receive a wire that helps to affix the bone plate to the bone.

9. The bone plate of claim 1, wherein the bone plate is composed of a biocompatible material.

10. The system of claim 1, further comprising:
a bone screw, comprising:
(a) a shaft portion for engaging the bone; and
(b) a head portion having a circular groove for engaging an aperture in a bone plate, the narrowest diameter of the circular groove being smaller than the diameter of the bone screw to either side of the circular groove.

11. The system of claim 10, wherein the shaft portion and/or head portion includes threads that terminate at the circular groove.

12. The system of claim 11, wherein the head portion is tapered to form a frustoconical section.

13. The system of claim 11, wherein the head portion includes an arcuate seating surface for further engaging the aperture.

14. The system of claim 13, wherein the arcuate seating surface is shaped to form a spherical radius.

15. The system of claim 13, wherein the arcuate seating surface is shaped to be complementary to a seating surface of the aperture.

16. The system of claim 11, wherein the circular groove lies in a plane substantially perpendicular to a long axis of the screw.

17. The system of claim 16, wherein the threads have a constant thread pitch.

18. The system of claim 11, wherein the circular groove engages the aperture by compressing a lip portion of the aperture within the groove.

19. The system of claim 10, wherein the bone screw is composed of a biocompatible material.

20. A bone screw, comprising:
a shaft portion for engaging the bone; and
a head portion having a circular groove for engaging an aperture in a bone plate, the narrowest diameter of the circular groove being smaller than the diameter of the bone screw to either side of the circular groove.
